(19) United States
(12) Patent Application Publication
(10) Pub. No.: US 2004/0176289 A1
(43) Pub. Date: Sep. 9, 2004

(54) Pharmaceutical composition for prevention and treatment of joint arthritis and a screening method thereof

(76) Inventors: Jang-Soo Chun, Kwangju (RU); Tae-Lin Huh, Daegu (KR)

Correspondence Address: MORGAN & FINNEGAN, L.L.P., 345 PARK AVENUE, NEW YORK, NY, 10154, US

(21) Appl. No.: 10/481,665

(22) PCT Filed: Jun. 17, 2002
(86) PCT No.: PCT/KR02/01138

(30) Foreign Application Priority Data
Jun. 19, 2001 (KR) 2001/34773

(51) Int. Cl. A61K 31/277; A61K 31/366

(52) U.S. Cl. 514/12; 514/469; 514/520

(57) ABSTRACT

The present invention relates to a pharmaceutical composition for prevention and treatment of arthritis and a screening method thereof. More precisely, the present invention relates to a pharmaceutical composition for prevention and treatment of arthritis which contains extracellular signal regulated protein kinase (ERK) or its upstream signal transduction molecule MAP kinase kinase (MEK) activity inhibitor, and a screening method for the pharmaceutical composition for prevention and treatment of arthritis through searching the ERK or MEK activity inhibitors.

### FIELD OF THE INVENTION

The present invention relates to a pharmaceutical composition for prevention or treatment of joint arthritis and a screening method thereof. More precisely, the present invention relates to a pharmaceutical composition for prevention or treatment of arthritis, which contains extracellular signal-regulated protein kinase (ERK) or its upstream signaling molecule such as mitogen-activated protein (MAP) kinase kinase (MEK) activity inhibitor, and a method for screening the pharmaceutical composition by searching for ERK or MEK activity inhibitors.

### BACKGROUND

Differentiated chondrocytes, which are the only cell type found in normal mature cartilage, synthesize sufficient amounts of cartilage-specific extracellular matrix (ECM) to maintain matrix integrity of articular cartilage. This homeostasis is destroyed in degenerative diseases, such as osteoarthritis and rheumatoid arthritis. Joint osteoarthritis is a disease that causes inflammation and destruction of articular cartilage. Although the molecular mechanisms of pathophysiology of arthritis are not completely characterized, arthritis is characterized by structural and biochemical changes in chondrocytes and cartilage, including degradation of the cartilage matrix via the accelerated activity of matrix metalloproteinase (MMP), insufficient synthesis of ECM due to loss of chondrocyte phenotype (i.e., de-differentiation) and increased cell death via apoptosis, and inflammatory responses of cartilage via induction of cyclooxygenase-2 (COX-2) expression. It is now generally accepted that pro-inflammatory cytokines, such as interleukin (IL)-1α and tumor necrosis factor (TNF)-α play a predominant role in structural and biochemical alterations in chondrocytes and cartilage during arthritis. The pro-inflammatory cytokines also increase the production of prostaglandin E2 (PGE2), leading to the inflammation of arthritis.

For the treatment of degenerative arthritis, therapeutic agents (anodynes, steroids, non-steroid anti-inflammation agents, etc.) or cartilage protecting agents (hyaluronic acid, glucosamine, chondroitin, etc.) have been used. If necessary, clinical operation (arthroscope operation, tibia proximal osteotomy, articular replacement, knee joint pre-replacement, etc.) has to be performed. The causes of arthritis (chondrocyte degeneration, MMP synthesis and activation, etc.), however, cannot be eliminated using these methods. For example, therapeutic agents merely alleviate pain and inflammation and require careful use due to such side effects as increased weight and the potential for inducing hypertension and digestive ulcers. Meanwhile, cartilage-protecting agents (e.g., hyaluronic acid, glucosamine, and chondroitin) merely supply nutrition to chondrocytes while protecting cartilage by relieving shocks. Such methods do not fundamentally treat arthritis caused by chondrocyte degeneration. Likewise, alternatives such as other medicines or surgery, though alleviating pain and inflammation, are not fundamental treatments.

So far, there has been no explanation of the mechanism by which the synthesis of cartilage matrix decreases by the de-differentiation or aging of chondrocytes. Although the generation of MMP, an articular matix lyase, induced by pro-inflammatory cytokine such as IL-1β and TNF-α, and the decomposition process of cartilage tissue thereby were known, the molecular regulation mechanisms such as the mechanisms of cytokine generation, gene expression, and protein activity regulation are still unclear. Therapeutic agents and operations for treatment of degenerative arthritis used these days can only relieve pain and inflammation. Hyaluronic acid, glucosamine, and chondroitin are not fundamental treatment agents for chondrocyte degeneration and arthritis thereby, but act simply as protective agents for chondrocytes.

Therefore, for a complete cure of arthritis, an explanation of several factors is required. These factors include (1) the mechanisms of MMP expression and activation, which are crucial factors for chondrocyte degeneration, (2) the mechanism which causes the decrease of generation of articular matrix in chondrocytes by a process of chondrocyte de-differentiation, (3) the process of signal transduction in chondrocytes, which is related to inflammation, the control system of gene expression, and the characteristics, and (4) the functions of related regulatory proteins.

The present inventors have confirmed that the activation of ERK or MEK stimulates the de-differentiation and degeneration of chondrocytes, increases the expression of MMP, which is a kind of protein lyase especially for decomposing articular cartilage tissue, and increases the expression of cyclooxygenase-2 (cox-2), which induces the production of PGE2 related with inflammation, resulting in the development and progression of arthritis. The present invention has been realized by confirming that the ERK or MEK activity inhibitor can be used as an effective treatment agent for arthritis by inhibiting the development and progression of arthritis.

### SUMMARY OF THE INVENTION

It is an object of this invention to provide a pharmaceutical composition for prevention or treatment of arthritis, which contains ERK or their upstream signal transduction molecule, MEK activity inhibitor as an effective ingredient.

It is a further object of this invention to provide a screening method for the pharmaceutial composition for the prevention and treatment of arthritis, by searching ERK or its upstream signal transduction molecule MEK activity inhibitor.

### BRIEF DESCRIPTION OF THE DRAWINGS

The application of the preferred embodiments of the present invention is best understood with reference to the accompanying drawings, wherein:

FIG. 1A is a schematic diagram showing the cell culture system for studying differentiation of chondrocytes;

FIG. 1B is a schematic diagram showing the cell culture system for studying de-differentiation of chondrocytes;

FIG. 2A is a chart including a set of photographs showing the differentiation of mesenchimal cells to chondrocytes by micromass culture;

FIG. 2B is a chart including a set of photographs and a graph showing the de-differentiation of chondrocytes by monolayer culture;

FIG. 3A is an electrophoresis photograph showing that the ERK activity inhibitor stimulates differentiation of chondrocytes by enhancing type-II collagen expression;

FIG. 3B is an electrophoresis photograph showing the inhibition of ERK activity by an ERK activity inhibitor;

FIG. 3C is a graph showing that the ERK activity inhibitor stimulates differentiation of chondrocytes;

FIG. 4A is a chart including a set of electrophoresis photographs showing that the ERK activity is increased during the de-differentiation process of chondrocytes;

FIG. 4B is a chart including a set of electrophoresis photographs showing that inhibiting ERK activity using an ERK activity inhibitor increases type-II collagen synthesis;

FIG. 4C is a chart including a set of electrophoresis photographs showing that the ERK activity inhibitor increases type-II collagen synthesis in a dose-dependent manner;

FIG. 4D is a chart including a set of graphs showing that the ERK activity inhibitor increases proteoglycan synthesis in a dose-dependent manner;

FIG. 5A is a chart including a set of photographs showing that the ERK activity inhibitor induces the increase of type-II collagen synthesis and the reduction of active ERK in the P0 and P2 stages;

FIG. 5B is a chart including a set of photographs showing that the ERK activity inhibitor induces the increase of type-II collagen synthesis and the reduction of active ERK in the P0 and P4 stages;

FIG. 6A is a graph showing the NO generation in chondrocytes treated with S-nitroso-N-acetyl penicillamine (SNP) in a dose-dependent manner;

FIG. 6B is a graph showing the NO generation in chondrocytes treated with SNP in a treating time-dependent manner;

FIG. 7A is a chart including a set of electrophoresis photographs showing the de-differentiation of chondrocytes treated with SNP by the decrease of type-II collagen expression;

FIG. 7B is a graph showing the de-differentiation of chondrocytes treated with SNP by the decrease of proteoglycan synthesis;

FIG. 8A is a chart including a set of electrophoresis photographs showing the pattern of ERK activation by the treatment time of SNP;

FIG. 8B is a chart including a set of electrophoresis photographs showing the pattern of ERK activation by the treatment concentration of SNP;

FIG. 8C is a chart including a set of electrophoresis showing that the expression of type-II collagen is recovered by the inhibition of ERK activity;

FIG. 8D is a graph showing that the expression of proteoglycan is recovered by the inhibition of ERK activity;

FIG. 9 is a chart including a set of electrophoresis photographs showing that the MMP-9 is activated by IL-1β;

FIG. 10A is an electrophoresis photograph showing that the ERK is activated by IL-1β;

FIG. 10B is an electrophoresis photograph showing that the IL-1β induced MMP-9 activation is restrained by the inhibition of ERK activity;

FIG. 11 is a graph showing that the IL-1β induced NO production in chondrocytes is restrained by the inhibition of ERK activity;

FIG. 12 is a chart including a set of electrophoresis photographs showing that the IL-1β induced cox-2 expression is restrained by the inhibition of ERK activity; and

FIG. 13 is a schematic diagram showing the effect of ERK on the differentiation and degeneration process of chondrocytes.

### DETAILED DESCRIPTION OF PREFERRED EMBODIMENTS

The present invention is based on the notion that the activation of ERK or MEK stimulates the de-differentiation and degeneration of chondrocytes, increases the expression of MMP, which is a kind of protein lyase especially for decomposing articular cartilage tissue, and increases the expression of cyclooxygenase-2 (cox-2), which induces the production of PGE2 related with inflammation, resulting in the development and progression of arthritis. The ERK or MEK activity inhibitor can be used as an effective treatment agent for arthritis by inhibiting the development and progression of arthritis.

Hereinafter, the present invention is described in detail.

In one aspect, the present invention provides a pharmaceutical composition for prevention or treatment of arthritis, which contains ERK or its upstream signal transduction molecule MEK activity inhibitor as an effective ingredient.

In a preferred embodiment, ERK or MEK activity inhibitors of the present invention include, but are not limited to, 2-(2-amino-3-methoxyphenol)-4H-1-benzopyrane-4-one (PD98059) and 1,4-diamino-2,3-dicyano-1,4-bis (2-aminophenylthio) butadiene (U-0126).

The criteria of ERK of the present invention include two types of ERK (ERK-1 and ERK-2) and every other ERK variant with more than 95% homology in their amino acid sequences with ERK-1 or ERK-2. The criteria of MEK of the present invention include two types of MEK (MEK-1 and MEK-2) and every other MEK variant with more than 95% homology in their amino acid sequences with MEK-1 or MEK-2. ERK is activated by MEK. Likewise, ERK activation is inhibited when MEK activity is prohibited, meaning MEK is the upstream signal transduction molecule of ERK.

An explanation of the operation of the pharmaceutical composition for prevention or treatment of arthritis that contains ERK or MEK inhibitor of the present invention is as follows.

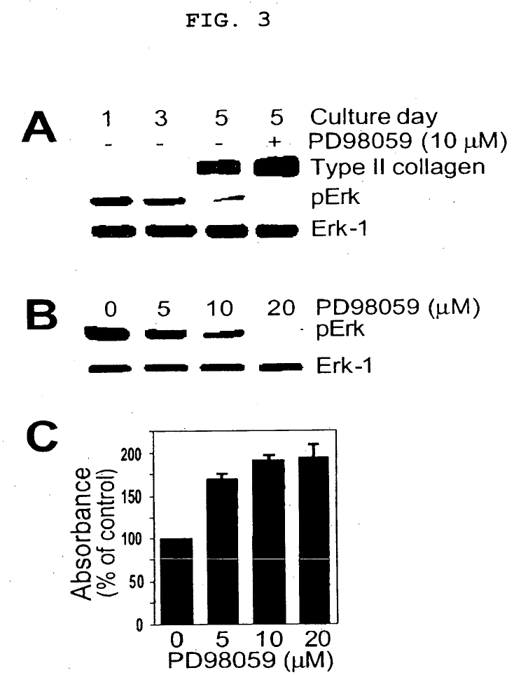

First, the present inventors investigated the effect of ERK or MEK activity inhibition on the differentiation of mesenchymal cells to chondrocytes. The differentiation to chondrocytes began from the third day of culturing, while mesenchymal cells of chicken embryo were sustained with multi-layer culture (see FIG. 3). At this time, the differentiation was demonstrated by confirming the expression of type-II collagen through Western blot analysis. There was no change of the ERK expression during the differentiation process of chondrocytes, but ERK activity was remarkably decreased, which is in contrast to the expression of type-II collagen. When the activation of MEK, the upstream signal transduction molecule of ERK, was inhibited by 2-(2-amino-3-methoxyphenol)-4H-1-benzopyran-4-one (PD98059), the ERK activity was decreased in a dose-dependent manner. At this time, proteoglycan synthesis in chondrocytes was confirmed to be remarkably increased through alcian blue staining, as was the expression of type-II collagen. Therefore, it has been proved that the inhibition of ERK or MEK activity is required for the differentiation from mesenchimal cells to chondrocytes. In other words, differentiation can be stimulated by inhibiting ERK or MEK activity.

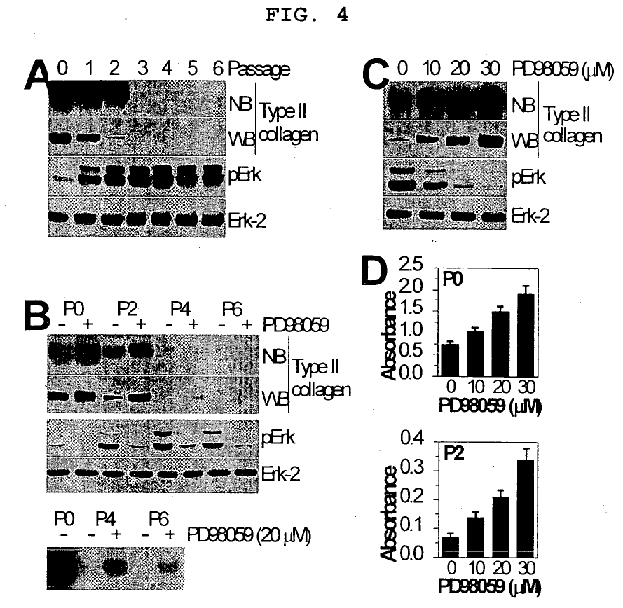

The present inventors studied the effect of ERK or MEK activity on de-differentiation taking place during the proliferation process of chondrocytes by monolayer culture in vitro. During the proliferation process of chondrocytes, the expression of type-II collagen was analyzed using Western (WB) and Northern (NB) blotting. As a result, the expression level was highest at P0, began to decrease at P1, and was not detected after P3 (see FIG. 4A). Meanwhile, the lowest ERK activity was shown at P0, but ERK activity increased by the following monolayer culture. Therefore, it was confirmed that ERK or MEK activity was in inverse proportion to the expression of type-II collagen, and ERK or MEK activity played an important role in de-differentiation accompanying the proliferation process of chondrocytes.

The present inventors also investigated type-II collagen and sulfated proteoglycan synthesis after treating the culture cells with ERK or MEK activity inhibitor in order to clarify the relation between ERK or MEK activity and de-differentiation. As a result, ERK activity was inhibited by the treatment of ERK or MEK activity inhibitor (see FIG. 4B) in a dose-dependent manner (see FIG. 4C). The expression of type-II collagen was remarkably increased in P0 cells where the ERK or MEK activity was inhibited, and the inhibited expression of type-II collagen turned to start expressing in P2 cells (see FIG. 4B). Along with the expression of type-II collagen, the inhibition of ERK or MEK activity stimulated proteoglycan synthesis by a multiplying factor of 2.6 in the P0 stage and by a multiplying factor of 6.4 in the P2 stage (see FIG. 4D). Therefore, the inhibition of ERK or MEK activity in de-differentiated chondrocytes was proved to induce the synthesis of type-II collagen and proteoglycan.

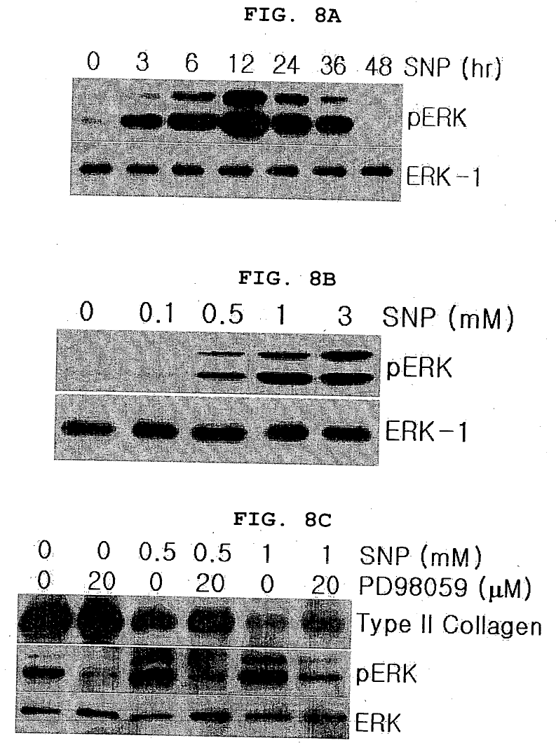

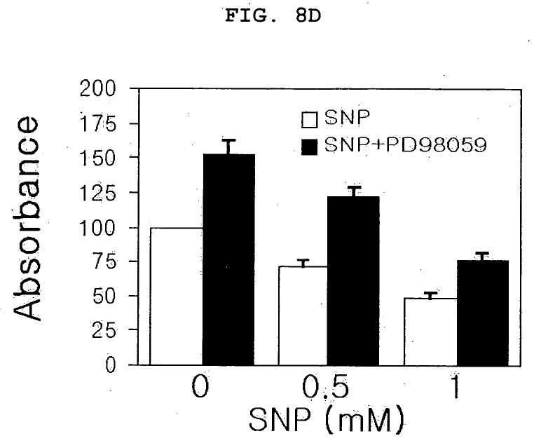

The NO generation caused by inflammatory cytokines in chondrocytes is accompanied by the destruction of cartilage matrix molecules. Again, the generated NO eventually induces production of enzyme, which destroys cartilage matrix and de-differentiation of chondrocytes. The present inventors confirmed that the generated NO by SNP treatment inhibited the expression of type-II collagen and proteoglycan, leading to the de-differentiation of chondrocytes (see FIG. 7). The NO generation in chondrocytes induced ERK activation in direct proportion to SNP concentration and treatment time. Meanwhile, the inhibition of ERK activity using an inhibitor was confirmed to restrain the de-differentiation of chondrocytes by NO (see FIG. 8). Therefore, the inhibition of ERK activity in chondrocytes was proved to stop the de-differentiation caused by NO and passaged culture. In other words, the inhibition of ERK activity could keep cartilage tissue stable by restraining the de-differentiation accompanied by arthritis.

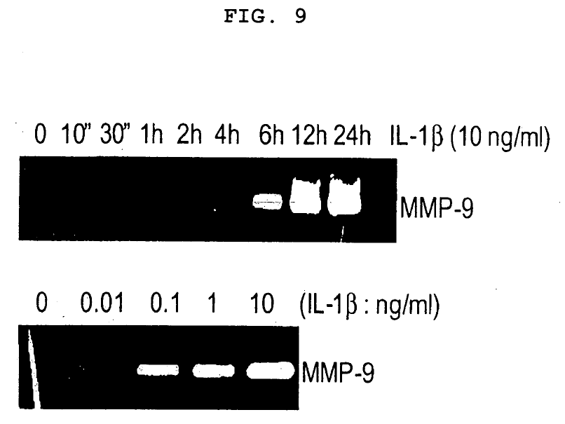

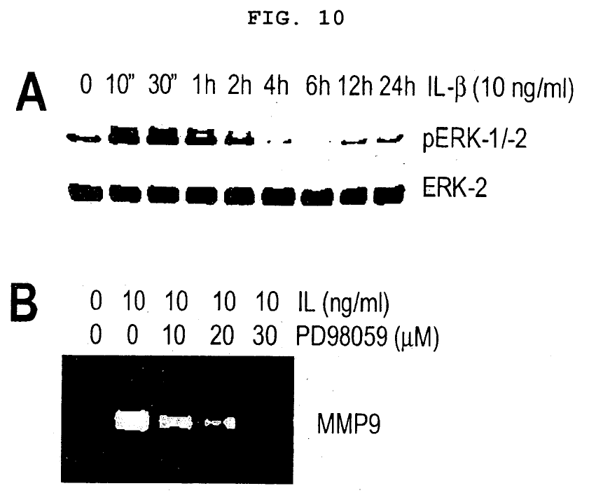

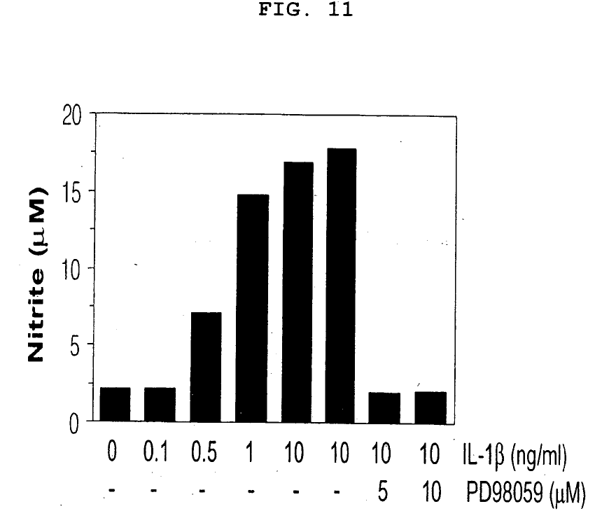

Considering that the destruction of cartilage matrix molecules is caused by the expression and activation of MMP by inflammatory cytokine, the present inventors have studied the relation between ERK activity and MMP expression. As a result, it was confirmed that the expression of MMP-9 was remarkably increased in proportion to the concentration of treated IL 1-β, an inflammatory cytokine (see FIG. 9). As chondrocytes were treated with IL 1-β, the ERK activity was temporary increased at the early stage, but when the ERK activity was inhibited by ERK activity inhibitor, the expression of MMP-9 was restrained (see FIGS. 10A and 10B). Therefore, the inhibition of ERK activity or its upstream signal transduction molecule MEK activity resulted in the increase of the synthesis of cartilage matrix molecule, which helped the formation and maintenance of cartilage tissue. The inhibition of MMP expression and activation in chondrocytes restrained the decomposition of cartilage matrix molecule, which also helped the maintenance of cartilage tissue.

In articular chondrocytes, IL-1β induced NO production in a dose-dependent manner. Thus, the present inventors have investigated the relation between the inhibition of ERK activity and the inhibition of NO production. As a result, it was confirmed that the NO production caused by IL-1β was completely blocked by inhibiting ERK activity using an ERK activity inhibitor (see FIG. 11). Arthritis worsens as MMP synthesis was increasingly induced by the generation of NO and pro-inflammatory cytokine. (Inflammatory cytokine induces NO generation, and the produced NO again induces the production of cytokine repeatedly.) Therefore, the inhibition of NO generation by inhibiting ERK or its upstream signal transduction molecule MEK activity can prevent the degeneration of chondrocytes, resulting in an effective use thereof for the treatment of arthritis.

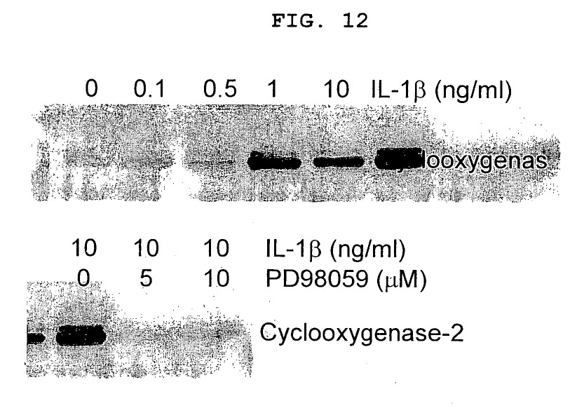

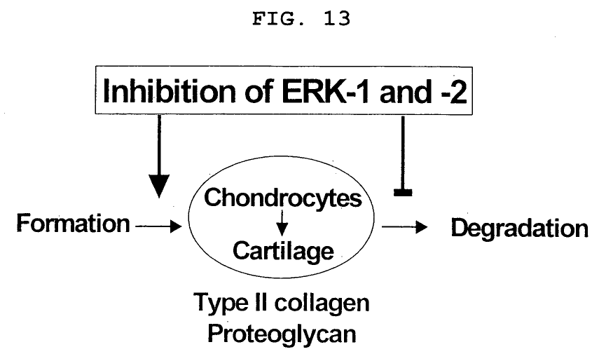

The present inventors have further investigated the relation between the expression of COX-2, a major mediator of inflammation in arthritis, and the inhibition of ERK activity. As a result, the expression of COX-2 was increased depending on the concentration of IL-1β treated in chondrocytes. The increase of COX-2 expression was totally restrained when the ERK activity was inhibited by the ERK activity inhibitor (see FIG. 12). Pro-inflammatory cytokine induces inflammation by producing PGE2, a kind lipid metabolite prostaglandin, via induction of COX-2 expression. Since PGE2 is produced by cox-2, the inhibition of cox-2 by blocking ERK activation suppresses inflammation. Therefore, the inhibition of COX-2 expression by ERK or its upstream signal transduction molecule MEK activity inhibitor contributes to the effective suppression of arthritis.

The pharmaceutical composition for prevention or treatment of arthritis, which contains ERK or its upstream signal transduction molecule, MEK activity inhibitor as an effective ingredient of the present invention can be administered orally or parenterally. The compound of the present invention can be prepared for oral or parenteral administration by mixing with generally used fillers, extenders, binders, wetting agents, disintegrating agents, diluents such as surfactant, or excipients. The present invention also includes pharmaceutical formulations in dosage units, which means that the formulations are presented in the form of individual doses, e.g., tablets, coated tablets, capsules, pills, suppositories, or ampules, the active compound content of which corresponds to one whole dose, a multiple of a whole dose, or the reciprocal (fraction) of the multple dose. Thus, the dosage units can contain, for example, one, two, three, or four individual doses or ½, ⅓, or ¼ part of an individual dose. An individual dose preferably contains a predetermined amount of active compound, which is administered in one application and which usually corresponds to one whole, one half, one third, or one quarter of a daily dose. Non-toxic inert pharmaceutically suitable excipients are to be understood as solid, semi-solid, or liquid diluents, fillers, and formulation auxiliaries of all types. Preferred pharmaceutical formulations include tablets, coated tablets, capsules, pills, granules, suppositories, solutions, suspensions and emulsions, pastes, ointments, gels, creams, lotions, dusting powders, and sprays. Solid formulations for oral administration include tablets, pill, dusting powders, and capsules. Liquid formulations for oral administrations include suspensions, solutions, emulsions, and syrups, and the above-mentioned formulations can contain various excipients such as wetting agents, sweeteners, aromatics, and preservatives in addition to generally used simple diluents such as water and liquid paraffin. Tablets, coated tablets, capsules, pills, and granules can contain the active compound or compounds in addition to the customary excipients, such as (a) fillers and extenders, for example, starches, lactose, sucrose, glucose, mannitol, and silicic acid; (b) binders, for example, carboxymethylcellulose, alginates, gelatine, and polyvinylpyrrolidone; (c) humectants, for example, glycerol; (d) disintegrating agents, for example, agar-agar, calcium carbonate, and sodium carbonate; (e) solution retarders, for example, paraffin; (f) absorption accelerators, for example, quaternary ammonium compounds; (g) wetting agents, for example, cetyl alcohol and glycerol monostearate; (h) adsorbents, for example, kaolin and bentonite; and (i) lubricants, for example, talc, calcium stearate, magnesium stearate, and solid polyethylene glycols; or mixtures of substances (a) to (i). The tablets, coated tablets, capsules, pills, and granules can be provided with the customary coatings and shells, optionally containing opacifying agents, and can also be of a composition such that they release the active compound or compounds only or preferentially in a certain part of the intestinal tract, if appropriate, in a delayed manner. Examples of embedding compositions, which can be used, would be polymeric substances and waxes. If appropriate, the active compound or compounds can also be presented in microencapsulated form with one or more of the above-mentioned excipients. Formulations for parenteral administration are stirilized aqueous solutions, water-insoluble excipients, suspensions, emulsions, and suppositories. Suppositories can contain, in addition to the active compound or compounds, the customary water-soluble or water-insoluble excipients, for example, polyethylene glycols, fats (e.g., cacao fat), and higher esters (e.g., C14-alcohol with C16-fatty acid), or mixtures of these substances. Ointments, pastes, creams, and gels can contain, in addition to the active compound or compounds, the customary excipients, for example, animal and vegetable fats, waxes, paraffins, starch, tragacanth, cellulose derivatives, polyethylene glycols, silicones, bentonites, silicic acid, talc, and zinc oxide, or mixtures of these substances. Dusting powders and sprays can contain, in addition to the active compound or compounds, the customary excipients, for example, lactose, talc, silicic acid, aluminum hydroxide, calcium silicate, and polyamide powder, or mixtures of these substances. Sprays can additionally contain the customary propellants, for example, chlorofluorohydrocarbons. Solutions and emulsions can contain, in addition to the active compound or compounds, the customary excipients, such as solvents, solubilizing agents, and emulsifiers, for example, water, ethyl alcohol, isopropyl alcohol, ethylcarbonate, ethyl acetate, benzyl alcohol, benzyl benzoate, propylene glycol, 1,3-butylene glycol, dimethylformamide, oils (in particular, cottonseed oil), groundnut oil, corn germ oil, olive oil, castor oil and sesame oil, glycerol, glycerol formal, tetrahydrofurfuyl alcohol, polyethylene glycols, and fatty acid esters of sorbitan, or mixtures of these substances. For parenteral administration, the solutions and emulsions are also be in a sterile form which is isotonic with blood. Suspensions can contain, in addition to the active compound or compounds, the customary excipients, such as liquid diluents, for example water, ethyl alcohol and propylene glycol, and suspending agents, for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances. The formulation forms mentioned can also contain coloring agents, preservatives and additives that improve the smell and taste, for example, peppermint oil and eucalyptus oil, and sweeteners, for example, saccharin. The above-mentioned pharmaceutical formulations can also contain other pharmaceutical active compounds in addition to the compounds according to the present invention. The above-mentioned pharmaceutical formulations are prepared in the customary manner by known methods, for example, by mixing the active compound or compounds with the excipient or excipients.

The therapeutically active compounds should preferably be present in the above-mentioned pharmaceutical formulations in a concentration of about 0.1 to 99.5% by weight of the total mixture, and preferably, about 0.5 to 95% by weight of the total mixture.

The formulations mentioned can be used on humans and animals orally, rectally, parenterally (intravenously, intramuscularly, or subcutaneously), intracisternally, intravaginally, intraperitoneally, or locally (dusting powder, ointment, or drops) and for the therapy of infections in hollow spaces and body cavities. Possible suitable formulations are injection solutions, solutions and suspensions for oral therapy and gels, infusiton formulations, emulsions, ointments or drops, ophthalmological and dermatological formulations, and silver salts. Other salts, eardrops, eye onintments, dusting powders, or solutions can be used for local therapy. In the case of animals, intake can also be in suitable formulations via the feed or drinking water. Gels, powders, dusting powders, tablets, delayed release tablets, premixes, concentrates, granules, pellets, boli, capsules, aerosols, sprays, and inhalants can furthermore be used on humans and animals. The compounds according to the present invention can moreover be incorporated into other carrier materials, such as, plastics (chain of plastic for local therapy), collagen, or bone cement.

In general, it has proved advantageous both in human and in veterinary medicine to administer the active compound or compounds according to the present invention in total amounts of about 0.001 mg/kg to about 100 mg/kg, preferably 0.01-10 mg/kg of body weight, one to three times every 24 hours, if appropriate, in the form of several individual doses, to achieve the desired results. However, it may be necessary to deviate from the dosages mentioned, and in particular to do so as a function of the nature and body weight of the object to be treated, the nature, and severity of the disease, the nature of the formulation and of the administration of the medicament, and the period or interval within which administration takes place. Thus, in some cases, it can suffice to manage with less than the above-mentioned amount of active compound, while in other cases the abovementioned amount of active compound must be exceeded. The particular optimum dosage and mode of administration required for the active compounds can be determined by any expert in the field.

The present invention also provides a screening method for a pharmaceutical composition for prevention and treatment of arthritis by searching ERK or its upstream signal transduction molecule MEK activity inhibitors.

As explained above, when the ERK or its upstream signal transduction molecule MEK activity is inhibited, the expression and activation of MMP, which is taking part in the decomposition of cartilage matrix are suppressed, NO production which makes chondrocyte degeneration worse by stimulating MMP synthesis is inhibited and the expression of cox-2 which induces the generation of PGE2 related with the inflammation of arthritis is also inhibited. Therefore, through searching ERK or MEK activity inhibitors, prevention or treatment agents of arthritis can be screened.

### EXAMPLES

Practical and presently preferred embodiments of the present invention are illustrated by way of the following Examples. It will be appreciated, however, that those skilled in the art, on consideration of this disclosure, may make modifications and improvements within the spirit and scope of the present invention.

### Example 1

### Differentiation and De-Differentiation of Chondrocytes

<1-1> Differentiation of Chondrocytes

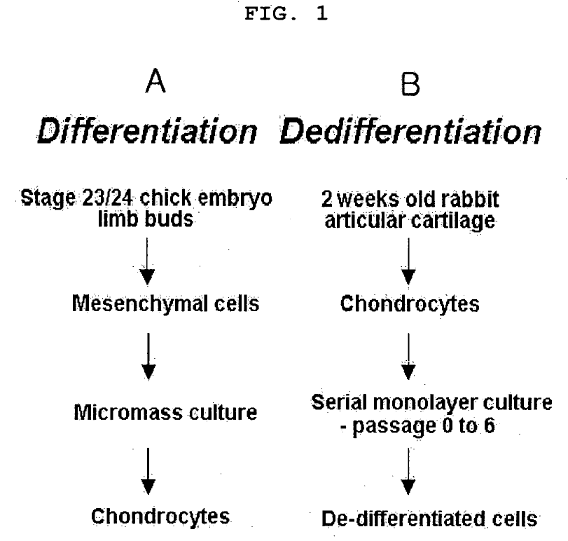

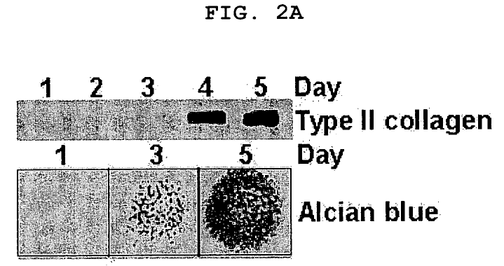

The present inventors have observed the differentiation process of chondrocytes using limb bud mesenchymal cells of Hamburger-Hamilton stage 24-25 chicken embryo under micromass culture (FIG. 1A). Particularly, mesenchymal cells were suspended in Ham's F-12 medium (Gibco BRL, Gaithersburg, Md., USA) supplemented with 10% fetal bovine serum (2×10⁷ cells/ ml). The cell suspension was loaded onto culture dish (15 μl/spot) and cultured at 37° C. for two hours in order to fix the cells on the dish. The cells were further cultured in the Ham's F-12 medium containing 10% FBS, 50 μg/ml streptomycin, and 50 units/ml penicillin.

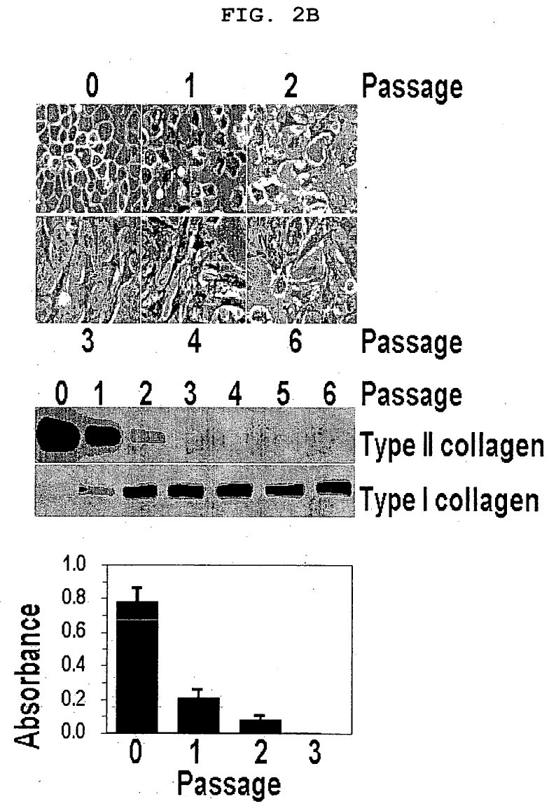

As a result, when mesenchymal cells were micromass cultured for five days, the synthesis of type-II collagen, a characteristic of chondrocytes, began to take place from the third day of culturing, which was confirmed by Western blotting using an antibody implying that the differentiation of chondrocytes was progressed. The accumulation of sulfated proteoglycan was also confirmed through alcian blue staining to be increased, which also implied that the differentiation of chondrocytes began from the third day of culturing and completed on the fifth day (FIG. 2A).

<1-2> De-differentiation of Chondrocytes

In order to confirm the de-differentiation and degeneration of chondrocytes, the present inventors have performed consecutively monolayer culture with articular chondrocytes of a two- to four-week-old rabbit (FIG. 1B). Particularly, articular cartilage of the rabbit was thin-sectioned aseptically, and the articular fragments were then reacted in PBS containing 0.2% collagenase type-II (Sigma) for six hours. Through quick-centrifugation, single cells were obtained. The single cells were suspended in DMEM (Dulbecco 's modified Eagle's medium, Gibco BRL, Gaithersburg, Md., USA) containing 10% FBS, 50 μg/ml streptomycin, and 50 units/ml penicillin and were plated onto a culture-dish at a density of 5×104 cells/cm² and cultured at 37° C. The medium was changed every other day. Five days later, the culture dish was filled with grown-up cells, which were identified as P0. Those cells were subcultured at the concentration of 5×104 cells/cm² per culture dish. The cells were continuously subcultured whenever they were fully grown to fill the dishes and were identified as P1 and next P2. Subculture was finished when P6 phase was completed. As a result, it was confirmed by Western blotting using antibodies that the expression of type-II collagen started to decrease at P0 and was totally restrained at P3. It was also confirmed by alcian blue staining that the accumulation of glycosaminoglycan sulfide was decreased as the subculture was continued. The de-differentiation and degeneration of chondrocytes were also disclosed by observing the shape of chondrocytes, which was changed from their distinguished round shape to fibroblast shape, a characteristic of degenerated chondrocytes (FIG. 2B).

### Example 2

### Stimulation of Chondrocyte Differentiation by the Inhibition of ERK Activity

The present inventors have confirmed the effect of ERK activity inhibition on the differentiation of chondrocytes. Particularly, the present inventors have investigated how the differentiation was influenced by the change and the inhibition of ERK activity during the differentiation process of chondrocytes induced by micromass culture of mesenchimal cells (FIG. 2A and Example 1-1). As a result, the differentiation was confirmed to start from the third day of culture while mesenchimal cells were kept under multi-layer culture, which was proved by confirming the expression of type-II collagen through Western blotting (FIG. 3A). During the differentiation process of chondrocytes, the expression of ERK was not changed, but the activity thereof was remarkably decreased unlike that of type-II collagen (FIG. 3A). When the activity of MEK, the upstream signal transduction molecule of ERK, was inhibited by treating 2-(2-amino-3-methoxyphenol)-4H-1-benzopyran-4-one (PD98059) in mesenchimal cells, the activity of ERK was decreased in a dose-dependent manner (FIG. 3B). At this time, the synthesis of proteoglycan was largely increased, which was confirmed by alcian blue staining (FIG. 3C), and the expression of type-II collagen was also increased (FIG. 3A). Therefore, it was confirmed that the decrease of ERK activity was required for the differentiation from mesenchimal cells to chondrocytes; that is, the differentiation of chondrocytes was stimulated by the inhibition of ERK activity.

### Example 3

### De-Differentiation of Chondrocytes by the ERK Activation

<3-1> ERK Activation and De-Differentiation of Chondrocytes

In order to ascertain the effect of ERK activity on the de-differentiation of chondrocytes, rabbit articular cartilage cells were cultured at the density of 5×104 cells/cm² on a culture dish. The cells began to proliferate on the second day of culture and reached saturation on the fifth day. The cells in saturation point were designated as P0. The cells in P0 stage had typical round or polygonal shapes, but these forms changed into flat and fibroblast-like shapes while the cells were going through P6 stage by serial monolayer culture (FIG. 2B). The expression of type-II collagen was analyzed by Western and Northern blotting, resulting in the highest expression in, P0, beginning to decrease in P1 and almost no expression after P3 (FIG. 4A). Meanwhile, the ERK activity was very low in P0 but remarkably increased by serial monolayer culture, which was not related to the expression of type-II collagen (FIG. 4A). Therefore, it was confirmed that the ERK activity was in inverse proportion to the expression of type-II collagen, which means the ERK activity plays an important role in the de-differentiation process.

<3-2> Suppression of De-Differentiation by the Inhibition of ERK Activity

To determine the relations between ERK activity and de-differentiation, the cells under subculturing were treated with 2-(2-amino-3-methoxyphenol)-4H-1-benzopyran-4-one (PD98059), an ERK activity inhibitor, and the synthesis of collagen and proteoglycan was observed. Particularly, 20 μM of ERK activity inhibitor, 2-(2-amino-3-methoxyphenol)-4H-1-benzopyran-4-one (PD98059), was added into chondrocytes of each passage and then cultured for two additional days, after which de-differentiation level was observed by examining the synthesis of collagen and proteoglycan. As a result, ERK activity was suppressed by the treatment of ERK activity inhibitor (FIG. 4B), and the inhibition level was depended on the concentration of treated compound (FIG. 4C). The expression of type-II collagen was largely increased in P0 cells wherein the ERK activity was suppressed. The inhibited expression of type-II collagen turned to express again in P2 cells (FIG. 4B). Along with the expression of type-II collagen, the inhibition of ERK activity increased the synthesis of proteoglycan by a factor of 2.6 in P0 and by 6.4 in P2, which was confirmed by alcian blue staining (FIG. 4D). Therefore, it was confirmed that the synthesis of proteoglycan was induced by the inhibition of ERK activity in de-differentiated chondrocytes.

<3-3> Expression and Distribution of Type-II Collagen Induced by ERK Activity Inhibition

In order to examine the suppression of de-differentiation by the inhibition of ERK activity, the expression of collagen was observed by immunostaining. Particularly, 20 μM of ERK activity inhibitor 2-(2-amino-3-methoxyphenol)-4H-1-benzopyran-4-one (PD98059) was added into P0 and P2 chondrocytes, and the cells were then cultured for two days. The cultured cells were fixed with 3% parpformaldehyde for ten minutes and some parts of the cell membranes were disrupted by 0.2% X-100. Next, anti-type II collagen antibody (10 μg/ml) was added thereto and continued to culture thereof for one hour. The secondary antibody conjugated with TRITC was added to those cells and cultured for one more hour, after which the expression and distribution of type II collagen were observed with fluorescence microscopy.

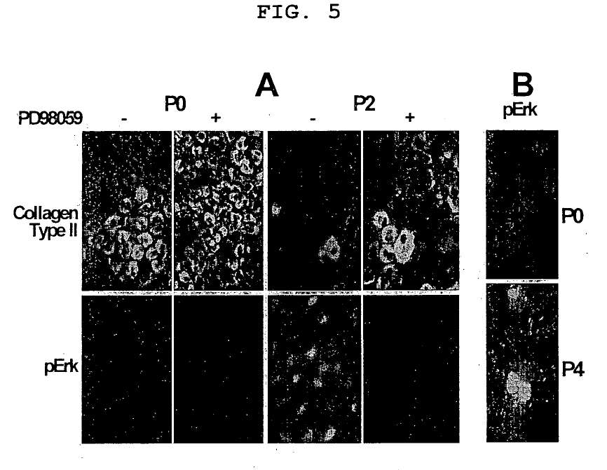

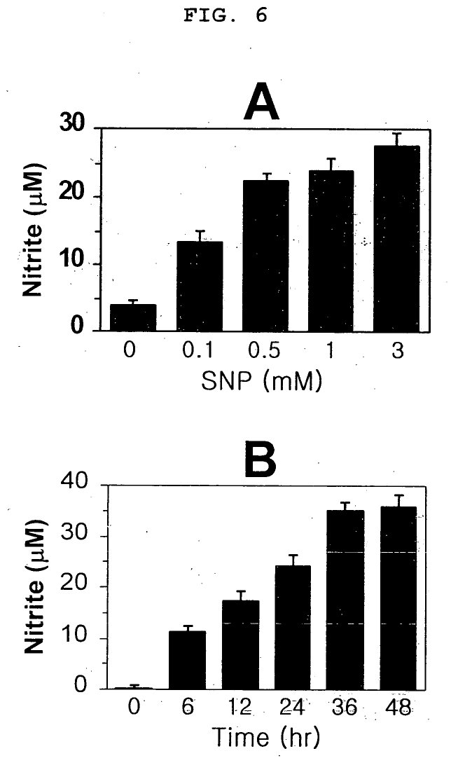

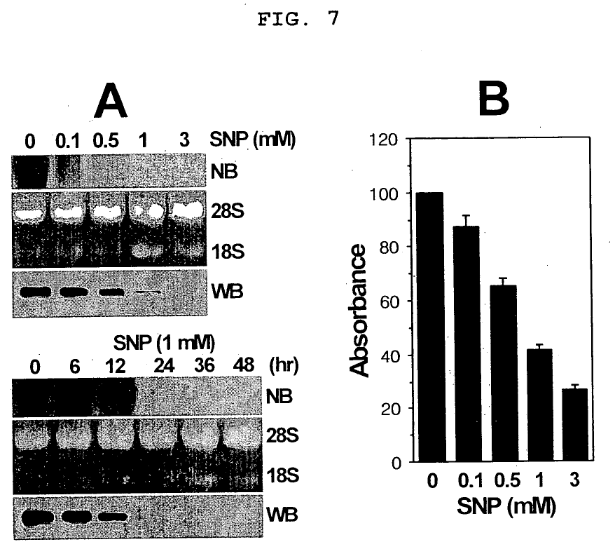

As a result, type-II collagen was heterogeneously expressed in most P0 cells (FIG. 5). When the cells were treated with ERK activity inhibitor, the numbers of type-II collagen expressed cells were remarkably increased. The extent of fluorescence representing the existence of type-II collagen was decreased in most P2 cells, but the numbers of type-II collagen expressed cells were greatly increased when the ERK activity was inhibited (FIG. 5). Therefore, it was proved that the inhibition of ERK activity induced more synthesis of collagen and proteoglycan in more cells, resulting in keeping cartilage tissue stable.

### Example 4

### Inhibition of NO Induced De-Differentiation of Chondrocytes by ERK Activity Inhibition

<4-1> NO Production in Chondrocytes

In order to investigate the effect of NO produced in chondrocytes on the de-differentiation of chondrocytes, the present inventors treated the cells with S-nitroso-N-acetyl penicillamine (SNP), a NO donor, to induce No production. As a result, it was confirmed that SNP treatment causes NO production in proportion to the concentration (FIG. 6A) and the treatment time of SNP (FIG. 6B).

<4-2> De-Differentiation of Chondrocytes by NO Production

It was confirmed by Western and Northern blotting that NO produced by SNP treatment in chondrocytes inhibited the expression of type-II collagen (FIG. 7A) and proteoglycan (FIG. 7B) in proportion to the concentration and the treatment time of SNP. When the cells were treated with SNP for 24 hours, the expression of type-II collagen was started to decrease in 0.1 mM-treated cells, and totally suppressed in 1 m M-treated cells. Meanwhile, when the cells were treated with 1 mM of SNP, the expression of type-II collagen began to decrease in 12-hour-treated cells, and totally suppressed in 24-hour-treated cells (FIG. 7A). The synthesis of proteoglycan was also confirmed to be decreased depending on the concentration of SNP (FIG. 7B), and thus NO produced by SNP was proved to induce de-differentiation of chondrocytes.

<4-3> Suppression of NO Induced De-Differentiation of Chondrocytes by the Inhibition of ERK Activity

In order to investigate the effect of ERK activity on the de-differentiation of chondrocytes induced by NO production, the change of ERK activity in SNP treated chondrocytes was detected. As a result, when the cells were treated with 1 mM of SNP, the ERK activity began to increase three hours after the treatment and reached the highest activity twelve hours later, but began to decreased 36 hours after the treatment (FIG. 8A). In order to increase the ERK activity by SNP, at least 0. 5 mM of SNP was required and the extent of ERK activity was in proportion to the concentration of SNP (FIG. 8B). It was also confirmed that ERK activity was totally restrained when 20 μM of ERK activity inhibitor, 2-(2-amino-3-methoxyphenol)-4H-1-benzopyran-4-one (PD98059), was added into chondrocytes. The ERK activity inhibitor also restrained the inhibition of type-II collagen expression by SNP (FIG. 8C), and it blocked the decrease of proteoglycan synthesis induced by SNP (FIG. 8D). Therefore, it was proved that the artificial inhibition of ERK activity using the inhibitor could suppress de-differentiation of chondrocytes induced by NO, resulting in keeping cartilage tissue stable.

### Example 5

### Suppression of MMP Expression by ERK Activity Inhibition

Considering the fact that the destruction of cartilage matrix molecules is caused by the expression and the activation of MMP by inflammatory cytokines, the present inventors detected the MMP-9 activity using gelatin zymography in order to prove the relation between the ERK activity and the MMP expression. Particularly, articular cartilage cells were treated with 10 ng/ml of IL-1β according to the different times (0 minutes, ten minutes, thirty minutes, one hour, two hours, four hours, six hours, twelve hours, and 24 hours) and concentrations (0 ng/ml, 0.01 ng/ml , 0.1 ng/ml, 1 ng/ml, and 10 ng/ml), and culture supernatant was then obtained, followed by electrophoresis on the gel containing 2% gelatin. The separated gel was cultured at 37° C. for twelve hours, by which the decomposition of gelatin was induced by MMP-9. The remaining gelatin was stained for the confirmation of gelatin decomposition by MMP, based on which the MMP-9 activity was measured.

As a result, the expression of MMP-9 was largely increased depending on the concentration of treated IL- 1β (FIG. 9). When chondrocytes were treated with IL- 1β, the ERK activity was temporarily increased at the early stage (FIG. 10A). The expression of MMP-9 was suppressed by the inhibition of the ERK activity using 10 μM of 2-(2-amino-3-methoxyphenol)-4H-1-benzopyran-4-one (PD98059) (FIG. 10B). Therefore, the inhibition of ERK activity was proved to enhance the formation and maintenance of chondrocytes by increasing the synthesis of cartilage matrix molecules and by prohibiting decomposition of cartilage matrix molecules through the inhibition of the expression and activation of MMP in chondrocytes.

### Example 6

### Suppression of NO Production by ERK Activity Inhibition

Considering that the IL- 1β stimulates NO production in a dose-dependent manner, the present inventors have investigated the relation between the inhibition of ERK activity and the suppression of NO production. Particularly, the amount of NO was determined by measuring the concentration of nitrite, a reaction product of NO, secreted after treating chondrocytes with IL1-β for 24 hours.

As a result, it was confirmed that NO production caused by IL-1β was completely inhibited by the inhibition of ERK activity using 5 μM of 2-(2-amino-3-methoxyphenol)-4H-1-benzopyran-4-one (PD98059) (FIG. 11). Arthritis worsens as the synthesis of MMP is increased by NO, which is produced by pro-inflammatory cytokine and the accompanying generation of self-productive cytokine by NO production. Therefore, the NO production inhibitor, which also inhibits ERK activity, can be effectively used for the prophylaxis and treatment of arthritis by preventing cartilage degeneration caused by inflammatory cytokine.

### Example 7

### Suppression of COX-2 Expression by ERK Activity Inhibition

The present inventors have further investigated the relation between the inhibition of ERK activity and the expression of COX-2. Particularly, the inventors have confirmed with Western blotting the quantity of cox-2 protein expressed in chondrocytes after treating the cells with different concentrations of IL-1β for 24 hours.

As a result, it was proved that the expression of cox-2 was increased depending on the concentration of treated IL-1β in chondrocytes but the expression was completely suppressed by the inhibition of ERK activity using 5 μM of 2-(2-amino-3-methoxyphenol)-4H-1-benzopyran-4-one (PD98059) (FIG. 12). Pro-inflammatory cytokines induce inflammation by generating PGE2, a kind of lipid metabolite prostaglandin. Since PGE2 is generated by COX-2, the suppression of COX-2 expression induced by the inhibition of ERK activity has the suppression effect of inflammation. Therefore, the COX-2 expression inhibitor based on the inhibition of ERK activity can be effectively used as an inhibitor of inflammation.

### Example 8

### Acute Toxicity Test in Rat Via Non-Oral Administration

The following experiments were performed to see if the compound of the present invention has acute toxicity in rat.

Six-week old SPF SD line rats were used in the tests for acute toxicity. 2-(2-amino-3-methoxyphenol)-4H-1-benzopyran-4-one (PD98059) of the present invention was suspended in 0.5% methyl cellulose solution and intravenous injected once into two rats per group at dosages of 50, 30, and 10 mg/kg/15 ml. Death, clinical symptoms, and weight change in the rats were observed, hematological tests and biochemical tests of blood were performed, and abnormal signs in the gastrointestinal organs of chest and abdomen were observed visually during autopsy. The results showed that the compound of the present invention did not cause any specific clinical symptoms, weight change, or death in rats. No change was observed in hematological tests, biochemical tests of blood, and autopsy. Therefore, 2-(2-amino-3-methoxyphenol)-4H-1-benzopyran-4-one (PD98059) of the present invention used in this experiment is evaluated to be safe substance since it do not cause any toxic change in rats up to the level of 50 mg/kg.

### Example 9

### Screening of Agents for Prevention and Treatment of Arthritis

For the activation of ERK, phosphorylation by MEK, the upstream signal transduction molecule of ERK, is required. As seen in FIGS. 3B, 4B, and 8C, the extent of ERK activity is confirmed with Western blotting using a phosphorylated ERK-specific antibody.

When the ERK or MEK activity is inhibited, the synthesis of cartilage matrix is increased, the expression and activation of MMP decomposing cartilage matrix is restrained, the production of NO making cartilage degeneration worse is prohibited, and the expression of COX-2 causing PGE2 generation related to inflammation of arthritis is also inhibited. Thus, it is confirmed that the ERK or MEK activity inhibitor can be used as a pharmaceutical composition for prevention and treatment of arthritis. The present inventors have also provided an effective screening method for a pharmaceutical composition for the prevention and treatment of arthritis by searching the ERK or MEK activity inhibitors.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. An agent for treatment and prophylaxis of arthritis containing as an effective ingredient extracellular signal-regulated protein kinase (ERK) or its upstream signal transduction molecule MAP kinase kinase (MEK) activity inhibitor.

2. The agent as set forth in claim 1, wherein the ERK is selected from the group consisting of ERK-1, ERK-2, and their variants with more than 95% homology in their amino acid sequences with ERK-1 or ERK-2.

3. The agent as set forth in claim 1, wherein the MEK is selected from the group consisting of MEK-1, MEK-2, and their variants with more than 95% homology in their amino acid sequences with MEK-1 or MEK-2.

4. The agent as set forth in claim 1, wherein the ERK or MEK inhibitor is selected from the group consisting of 2-(2-amino-3-methoxyphenol)-4H-1-benzopyran-4-one (PD98059) and 1,4-diamino-2,3-dicyano-1,4-bis (2-aminophenylthio) butadiene (U-0156).

5. The agent as set forth in claim 4, wherein the ERK or MEK inhibitor is 2-(2-amino-3-methoxyphenol)-4H-1-benzopyran-4-one.

6. A screening method of an agent for treatment and prophylaxis of arthritis through searching ERK or its upstream signal transduction molecule MEK activity inhibitor.

7. The screening method as set forth in claim 6, wherein the ERK is selected from the group consisting of ERK-1, ERK-2, and their variants with more than 95% homology in their amino acid sequences with ERK-1 or ERK-2.

8. The screening method as set forth in claim 6, wherein the MEK is selected from the group consisting of MEK-1, MEK-2, and their variants with more. than 95% homology in their amino acid sequences with MEK-1 or MEK-2.
